(19) United States
(12) Patent Application Publication
(10) Pub. No.: US 2008/0027320 A1
(43) Pub. Date: Jan. 31, 2008

(54) Multidimensional transducer systems and methods for intra patient probes

(75) Inventors: Mirsaid Bolorforosh, Portola Valley, CA (US); David I. Bruce, Lafayette, CA (US); Stephen R. Barnes, Bellevue, WA (US); Todor Sheljaskow, Issaquah, WA (US); Jian-Hua Mo, Milpitas, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS USA, INC.

(21) Appl. No.: 11/904,944

(22) Filed: Sep. 27, 2007

Related U.S. Application Data
(62) Division of application No. 10/848,855, filed on May 18, 2004.

(51) Int. Cl. A61B 8/00

(52) U.S. Cl. 600/439

(57) ABSTRACT

Endocavity and invasive catheter transducers for four-dimensional or other imaging are provided. A two-dimensional or other multi-dimensional array of elements is connected with a minimum number of conductors to an imaging system. One or more conductors are used to select an aperture, such as selecting one or more rows of elements for activation. Along a different axis, such as an orthogonal axis, elements are used to image a planar region. By electronically switching the selected aperture, different planes are rapidly imaged. A matrix configuration of electrodes, such as using column electrodes for phased array imaging and row electrodes for selecting an elevation aperture allows for rapid acquisition of ultrasound data.

### REFERENCE TO RELATED APPLICATIONS

The present patent document claims the benefit of the filing date pursuant to 35 U.S.C. §119(e) of Provisional U.S. Patent Application Ser. No. 60/527,144, filed Dec. 5, 2003, which is hereby incorporated by reference.

### BACKGROUND

The present invention relates to intra-patient probes. In particular, transducers and associated methods for acoustically imaging with an intra-patient probe are provided.

Intra-patient probes include endocavity probes, such as transesophageal, rectal or vaginal probes. Intra-patient probes also include intra-vascular and intra-cardiac catheters. The catheter is inserted within the venous or arterial system by puncturing one or more tissues on a patient.

To assist in medical examination, diagnosis or procedures, a transducer array is provided on the intra-patient probe. For example, a linear array, a phased array or a multi-dimensional array is provided for generating an ultrasound image. Linear or phased arrays generate an image representing a planar region running parallel to the array, such as a cross section or a longitudinal view of a vessel or organ. A multi-dimensional array may provide for multiple views, such as using three linear arrays configured in an “I” pattern to generate three planar images. However, planar images may provide limited context information, resulting in difficulty in identifying a current position of the intra-patient probe or region being imaged. Intra-patient probes may have limited space for connecting ultrasound transducers with an imaging system. Such connections are typically performed with coaxial cables or other conductors, one for each element of an array. Catheters in particular have very limited space given the small diameter typically used.

To provide better contextual information, planar information may be used to generate a three-dimensional image. Signal processing or other techniques for identifying the location associated with each planar image is used to reconstruct a three-dimensional volume representation from a plurality of scans as the intra-patient probe is moved. However, such processes rely on a static environment. Many organs and other structures within a patient move in response to one or more of various cycles, such as the breathing or heart cycle. As a result, static information may be inaccurate and undesired.

### BRIEF SUMMARY

By way of introduction, the preferred embodiments described below include methods and systems for imaging with intra-patient probes. Endocavity and invasive catheter transducers for four-dimensional or other imaging are provided. A two-dimensional or other multi-dimensional array of elements is connected with a minimum number of conductors to an imaging system. One or more conductors are used to select an aperture, such as selecting one or more rows of elements for activation. Along a different axis, such as an orthogonal axis, elements are used to image a planar region. By electronically switching the selected aperture, different planes are rapidly imaged. A matrix configuration of electrodes, such as using column electrodes for phased array imaging and row electrodes for selecting an elevation aperture allows for rapid acquisition of ultrasound data in different planes.

In a first aspect, a transducer is provided for use in an intra-patient probe. A multi-dimensional array of elements connects with an intra-patient probe housing. First electrodes extend over at least two elements along a first axis. Second electrodes extend over at least two elements along a second axis different than the first axis.

In a second aspect, a transducer is provided for use in an intra-patient probe. A multi-dimensional N×M array of elements connects with an intra-patient probe housing. N and M are either equal or different and both greater than 1. Switches are operable to connect a voltage source to one or more selected electrodes. One of a transmitter and receiver is connectable with other electrodes. The other electrodes form a phased array with an elevation extent corresponding to the electrodes connected with the first voltage source.

In a third aspect, a method is provided for imaging with a multi-dimensional array of an intra-patient probe. A first group of elements of a multi-dimensional array is activated. Ultrasound data is acquired with the first group of elements during the activation. A second group of elements different than the first group is activated where at least one element is active during one activation and inactive during the other activation. Further ultrasound data is acquired with the second activation. An image is generated as a function of the ultrasound data acquired with the different activations.

In a fourth aspect, a transducer is provided for use in an ultrasound system for medical imaging or therapy. A two-dimensional ultrasonic acoustic array mounts on a catheter. Switches are operable to apply a voltage to selectively activate array elements. At least one array element is free of activation while at least one other element is activated.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a perspective cut away diagram of one embodiment of an intra-patient probe with a multi-dimensional array;

FIG. 2 is a block diagram showing one embodiment of a matrix structure for operating a multi-dimensional transducer array;

FIG. 3 is a circuit diagram showing an interconnection of circuits to an element in one embodiment; and

FIG. 4 is a flow chart diagram showing one embodiment of a method of acquiring ultrasound data with a multi-dimensional array of an intra-patient probe.

### DETAILED DESCRIPTION OF THE DRAWINGS AND PRESENTLY PREFERRED EMBODIMENTS

A matrix arrangement of electrodes and associated connections with an imaging system are provided to reduce the number of conductors connected with a multi-dimensional array in an intra-patient probe. For example, one set of electrodes extend in parallel along an entire azimuth extent of an array for selectively actuating an elevation aperture. Another set of electrodes extend in parallel along an entire elevation extent of an array for operating as a phased array structure along the azimuthal dimension in the activated aperture. By walking or moving the selected aperture to different rows, the column electrodes may be used as a linear or phased array for imaging at different planar slices of a three-dimensional volume. Other matrix configurations of the electrodes and associated electronics may be provided.

Due to the limited size available in a catheter, a limited number of conductors for connecting a multi-dimensional array to an imaging system are provided. The matrix configuration allows an active aperture to electronically move along an elevation plane to produce ultrasound beams that may interrogate an entire volume. In one example embodiment, the multi-dimensional array is a 32×32 arrangement of elements. Rather than providing 1,024 conductors for the electronic steering in both elevation and azimuth dimensions, 32 conductors and associated electrodes are used to define an aperture and another 32 electrodes and associated conductors are used for two-dimensional imaging using the defined apertures. Sixty four total conductors may be better suited for an intra-patient probe, such as an intra-vascular catheter with a dimension of 8 to 12 French, to fully utilize the available space inside the catheter

FIG. 1 shows one embodiment of a cut-away view of a transducer system 10 for use in an intra-patient probe. The transducer system 10 includes an intra-patient probe housing 12, a handle 14 and a multi-dimensional array 16. Additional, different or fewer components may be provided. The transducer system 10 connects directly or indirectly through coaxial cables or other conductors to an imaging system for generating one, two or three-dimensional representation. The connection is permanent or the transducer system 10 may be disconnectable.

As shown in FIG. 1, the intra-patient probe housing 12 is a cardiac catheter housing of less than 15 French in diameter. For example, the probe housing 12 is an elongated flexible tube, 8 to 12 French in diameter, for insertion into a vascular system of a patient. Any now known or later developed materials, such as bio compatible polymers, may be used as the housing. The material is sufficiently flexible to allow insertion and guidance through the vascular system. Guide wires, stiffening inserts, other tubes, ports, lumens or other now known or later developed catheter components may be included within, on or as part of the catheter housing 12.

In other embodiments, the intra-patient probe housing 12 is an endocavity, vaginal, rectal, transesophageal, intra-operative, laparoscopic or other now known or later developed ultrasound transducer probe for insertion within a patient. While shown as cylindrical in FIG. 1, the probe may have any of various now known or later developed shapes, such as bulbubous, cubical, flat, rounded or other shapes. Endocavity and intra-operative probes are rigid, but may include steerable, bendable or otherwise guidable sections. For example, a transesophageal probe includes a transducer array 16 that is rotatable about one axis as well as a bendable portion of the shaft. The length of the housing 12 is adapted to the use, such as having a shorter length for intra-operative or endocavity probes than for a catheter.

In one embodiment, a position sensor is within the catheter. For example, a magnetic, gyroscope, strain gauge or other position sensor is provided within the catheter, such as along the axis of the housing 12 or adjacent to the array 16, to determine a position of the array 16. The sensed position may be used for forming three-dimensional images as the array 16 is moved within the patient.

A single array 16 is shown on the housing 12. In alternative embodiments, a plurality of arrays 16 is provided on the housing 12. For example, a plurality of multi-dimensional arrays 16 as described herein is provided. As another example, both multi-dimensional and one dimensional arrays are provided. The arrays 16 are spaced around a circumference, along a length or axis, at a tip and spaced away from the tip, at other relative positions along the housing 12, or combinations thereof.

The multi-dimensional array 16 is an array of elements 20 connected with the housing 12. Connected with is used herein to include direct or indirect connection, such as being connected to interior components indirectly connected with the housing 12. The array 16 is positioned on top of, adjacent to, or under the housing 12. For example, an acoustic window is provided as integrated with the housing 12 and positioned over the array 16. Alternatively, the material of the housing 12 is acoustically transparent or sufficiently transparent to allow imaging through the material without a separate window.

The elements 20 of the acoustic array 16 are elecstrostrictor materials such as PMN-PT, piezoelectric (PZT), capacitive micro machined membrane ultrasound transducers (CMUT) or other now known or later developed material for transducing between acoustic and electrical energies. Composites, such as 1-3 composites, of PZT or electrostrictor materials may be used to allow curvature. Any of various electrostrictor materials may be used, such as an electrostrictor ceramic of relaxer ferroelectric material. The electrostrictor ceramics have a depolarization temperature that is close to room or patient temperature (e.g., −10 Deg. C. up to 70 Deg. C.). The random polarization of the electrostrictor ceramic results in an inert material for transduction at or above the depolarization temperature. By applying a polarization voltage at or above the depolarization temperature, the material becomes active due to the polarization alignment in the material. As a result, a polarization voltage may activate or a lack of voltage may inactivate the electrostrictor ceramics.

Capacitive membrane transducers are formed with CMOS or semiconductor processes and materials to generate one or more membranes with an associated gap for each element. The flexing of the membrane with associated electrodes allows for transducing between acoustical and electrical energies. For curved capacitive membrane based transducers, the silicon or other substrate is thinned and curved on an appropriate support structure, such as a backing block. Alternatively, discrete segments are positioned adjacent to each other to form a substantially curved surface. In a third alternate, a curved CMUT may be formed directly on the surface of a silicon or other suitable cylindrical substrate. A bias voltage is typically applied to the membranes. By increasing a bias voltage, a membrane may be bottomed out, preventing or minimizing movement in response to radiofrequency, transmission electric signals or acoustic reception signals. An increase in bias voltage may be used to deactivate the membrane based transducer elements.

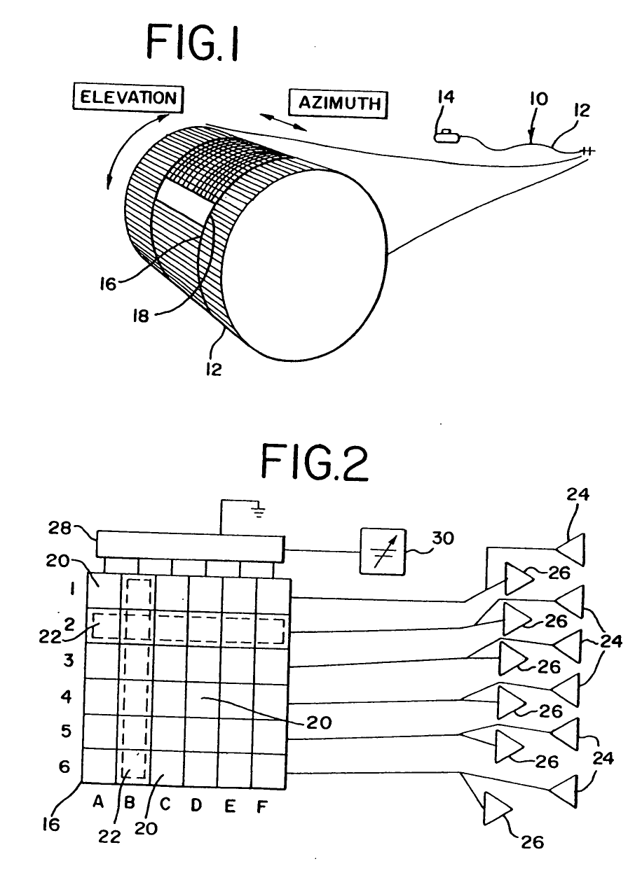

As shown in FIG. 2, the multi-dimensional array 16 is an N×M array of elements 20. N and M are either equal or different, such as a 32×32, 64×12 or a 40×20 array. The RF signal is applied along one axis and an activation polarization or bias voltage is applied along another axis, such as an orthogonal axis, plane to selectively control the active region along the other plane. In one embodiment, the elements 20 are distributed in rows, such as labeled A through F in FIG. 2, and columns, such as labeled 1 through 6 in FIG. 2. The elements 20 are distributed on a rectangular or square grid pattern, but hexagonal, triangular or other now known or later developed grid patterns with full or sparse sampling may be used. In another embodiment, the array 16 is an annular or sector array. The elements 20 are acoustically isolated from each other by kerfs or gaps filled with air, epoxy, gas, polymer or other now known or later developed materials. In alternative embodiments, the elements 20 are defined by an intersection of electrodes or placement of electrodes without kerfing. Elements 20 with a square, rectangular, hexagonal, triangular or other shape may be provided.

As shown in FIG. 2, the array 16 has four edges as part of a rectangle or square. For a hexagonal configuration, six edges may be provided. For a triangular configuration, three edges may be provided. In yet other embodiments, the number of edges is different than the grid pattern or element shape. The edges or the outermost elements conform directly to or generally follow over multiple elements the edge of the array 16. Any now known or later developed shapes may be used.

As shown in FIG. 1, the array 16 is concave from the perspective of within the catheter and convex from the perspective of the exterior of the catheter or other housing 12 for conforming to the cylindrical outer surface of the housing 12. In one embodiment for use in a catheter housing with 12 French diameter or less, a radius of curvature of 4.537 millimeters over a 45 degree viewing angle is provided. Seven individual rows or segments together extend over 1.4 millimeters (e.g. 0.2 mm per segment) for imaging at 4 cms of depth. In alternative embodiments, a flat or convex curvature is provided. Combinations of concave, convex and flat curvature may be use in other alternative embodiments. The array 16 shown in FIG. 1 is concave along one dimension and flat on another dimension. In alternative embodiments, a spherical or other curvature applied along more than one dimension is provided, such as conforming the array 16 to a tip of the housing 16. The array 16 shown in FIG. 1 extends around only a portion of a circumference of the housing 12, such as around a 45 to 90 degree angle of the circumference. In alternative embodiments, a lesser or greater extent is provided, including extending around the entirety of the circumference 12 to form a cylindrical shaped array.

Two sets of electrodes 22 are provided on two different sides of the array 16 as shown in FIG. 2. One set of electrodes 22 extends over at least two elements along a first axis. For example, the six electrodes extend along the columns A through F from the row labeled 1 to the row labeled 6 or between opposite edges. The second set of six electrodes 22 extends along the rows 1 through 6 from elements A through F or between two other opposite edges. Dashed lines labeled 22 represent two orthogonal electrodes in FIG. 2. As a result, the electrodes of one set of electrodes 22 extend over at least two elements along one axis, and the electrodes of the other set of electrodes 22 extend at least along two elements along a second axis. As shown in FIG. 2, the axes are orthogonal to each other. The first set of electrodes is connected to RF transmitters and receiver preamps along the azimuth plane. The second set of electrodes is connected along the elevation plane forming to the bias control circuit. The second set of electrodes selectively selects the appropriate aperture along the elevation plane. By moving the active aperture along the elevation plane, imaging data can be acquired throughout the entire volume allowing three dimensional ultrasound imaging. In order to increase the three dimensional imaging field of view in one mode of operation, the array is curved along the elevation plane. Alternatively, different angles may be provided, such as associated with the triangular or hexagonal grid pattern (e.g., 60 or 67 degrees). By extending from opposite edges, electrodes of one set of electrodes 22 extend along rows to a greater extent than along columns, and the electrodes of the other set of electrodes 22 extend along columns to a greater extent than along rows. In alternative embodiments, any of the rows or column extent of the electrodes 22 may be more limited, such as providing two separate electrodes 22 to extend along three elements in FIG. 2 along a same row or column. The use of an axis or axes herein includes accounting for any curvature of the array. For example, the axes are considered orthogonal to each other for the rectangular grid of array 16 of FIG. 2 as the concave array 16 shown in FIG. 1. One of the axes curves with the concavity of the array.

By positioning the electrodes 22 on opposite surfaces the elements 20 and associated array 16, both sets of electrodes 22 cover all of the elements or generally extend to each of the edges of the array 16 or close to the edges of the array 16. As a result, each element 20 is associated with a different electrode 22 on a top and bottom surface. One set of electrodes 22 is for applying a radiofrequency transmission signal or receiving signals generated in response to acoustic echoes. The other set of electrodes 22 on an opposite surface is used for applying a desired DC bias or other signal to activate or deactivate selected elements 20.

For a capacitive membrane ultrasound transducer, the electrodes adjacent to the membrane, such as on a top surface above the membrane are interconnected using switches, relays or deposited conductors. The electrodes associated with the gap are then interconnected through doping, depositing or other formation of electrical interconnections between desired membrane cells to form elements and electrodes. Since any of various patterning may be used in the formation of a capacitive membrane ultrasound transducer, the axes associated with the electrodes on the top and bottoms of the elements may be at any selected angle to each other and may vary in angle along the extent of the array.

For an annular or sector array, one set of electrodes forms annular rings on one surface and the other set of electrodes form pie shaped wedges orthogonal to the annular rings. Different pie shaped wedges, such as a pair of mirror image sectors, are activated for imaging, providing a bow tie shaped phased array for forming images in a plane normal to the array. The array may or may not include a center or bulls eye element.

The electrodes 22 are positioned with the array 16 relative to the housing 12. For example, a convex cylindrical array 16 with electrodes 22 on one surface is oriented parallel to an axis of curvature and the electrodes on the opposed face are oriented orthogonal to the axis of curvature. A cylindrical image may then be walked orthogonal to the axis of curvature, sweeping out a three-dimensional volume with acoustic scans. Alternatively, the image plane in phased array scans are formed in parallel to the long axis of the cylinder and the associated aperture is sequentially positioned angularly around the axis to sweep out different image planes. Linear array beamforming or phased array beamforming may be used.

Conductors, such as wire bonds, flex circuits, connection pads or other conductors extend from the edges or surfaces of the array 16. As shown in FIG. 2, conductors for one set of electrodes 22 extend from one edge and conductors for another set of electrodes 22 extend from an adjacent edge. In other embodiments, the conductors extend from opposite edges for each set of electrodes. In yet other embodiments, the conductors associated with each set of electrodes extend from a same edge of the array 16. For a capacitive membrane ultrasound transducer, separate traces or signal tracks for different electrodes may be formed on a same surface. In one embodiment, the conductors of the electrodes 22 connect to a two-layer flex foil bonded to the array matrix. Vias, patterning, edging and combinations thereof are used to maintain individual electrodes 22 as separate from electrodes 22 of the same set or different set. The void or gap between the membrane electrode and another electrode also acts to isolate signals for the different sets of electrodes.

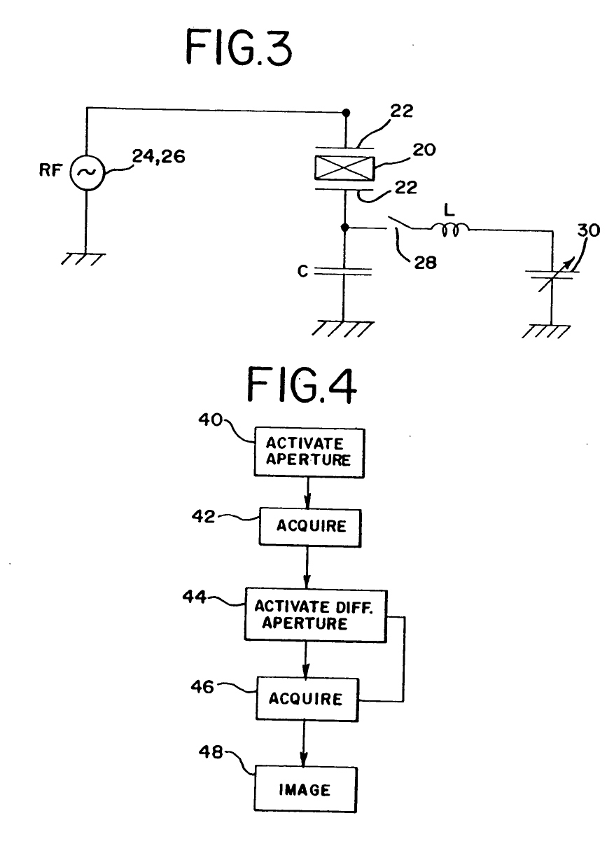

FIG. 3 shows one embodiment of a circuit configuration for shunting an electrode 22 to ground during use by transmitters or receivers 24, 26. The capacitors and inductors act to shunt an electrode 22 to ground with or without the application of a DC bias through the switch 28 to the DC source 30. To minimize cross-coupling between active and inactive elements 20, capacitors and back-to-back diodes or mirror diodes connect between each electrode 22. Dicing may alternatively or additionally minimize cross coupling.

Referring again to FIG. 2, an arrangement of transmitters 24, receivers 26, switches 28 and one or more DC voltage sources 30 for use with the array 16 and associated electrodes 22 is shown. Additional, different or fewer components may be provided, such as providing multiple voltage sources 30, providing transmitters 24 without receivers 26, providing receivers 26 without transmitters 24, providing the switches 28 within the array 16 or combinations thereof.

The voltage source 30 is a DC voltage source for providing a selectable amount of constant or variable bias voltage. The voltage source 30 is a voltage divider, a plurality of voltage dividers with associated transistor switches for selecting a voltage, a digital-to-analog converter, a transformer, a relay, a switch network or other now known or later developed voltage source. In one embodiment, a single voltage source 30 is provided for outputting a single voltage. In other embodiments, a plurality of voltage sources 30 are provided for outputting different voltages, such as one voltage source for outputting a first voltage and a second voltage source for outputting a greater voltage. One or more voltage sources may also be used for driving a voltage to ground, such as having a switch selecting between a positive or negative voltage and a ground potential. Any of the bias voltages and/or components disclosed in U.S. Pat. No. ______ (Publication No. 2003/0048698), the disclosure of which is incorporated herein by reference may be used.

The voltage source 30 is operable to activate a selectable aperture of less than all of the elements 20 of the array 16. For example, a bias voltage is provided to some but not all of the elements 20 through selection of column or row electrodes 22 to be connected with the voltage source 30 as opposed to a ground. For electrode-restrictive elements, the bias voltage activates the elements for transducing between acoustical and electrical energies. Elements 20 without bias voltage remain inert or deactivated. As another example, one bias voltage is provided for activating some elements of a CMUT, and a greater bias voltage is applied to other elements 20 at a same time to bottom out the membranes, inactivating the elements 20. As yet another example, some elements are selected for connection to ground for activation while other elements are selected for connection to a AC voltage source, such as one or more of the transmitters 24 for inactivation by applying a same signal to both electrodes 22 of the elements 20.

In one embodiment, multiple voltage sources 30 are provided for applying apodization. The apodization controls the imaging beam side lobe level. The apodization function can be positive or negative to optimize the beam profile. The beam profile optimization is a trade off between beam width at the top (0-10 dB) and the lower (10-40 dB) portions of beam width. For example, a center column of elements 20 has a relative bias voltage such that the relative response strength to a radio frequency (rf) signal is one and other columns, such as two outer columns of elements 20 adjacent to the center column of elements 20, has a higher bias voltage such that the relative response strength to a same rf signal is 1.67. As another example, two center segments have a 1 weighting and two outer segments in a four column aperture have 1.59 relative weighting. As yet another example in a five column aperture, the three center columns have a relative one weighting and the outer two columns have a 1.67 weighting. As yet another example embodiment with a six column aperture weighting, a relative 1.0 weighting is providing for the center two columns and a 1.64 relative weighting is provided on the outer four columns. Other relative weightings using 2, 3 or more bias voltages may be provided. As yet another alternative embodiment, the bias voltages for a multi-segment aperture have a same weighting. The relative weighting may relate linearly or non-linearly to the associated applied bias voltages.

A plurality of switches 28 is operable to connect the voltage source 30 to one or more selected electrodes 22. The switches 28 are transistors, relays, multiplexer, switch network, digital devices, analog devices, application specific integrated circuit, combinations thereof or other now know or later developed devices for selectively connecting different voltage sources or ground potential to different electrodes 22. In one embodiment, the switches 28 are connected with the electrodes 22 through a flexible circuit or other conductors. In an alternative embodiment, one or more of the switches 28 are formed as part of the array 16, such as integrated with a capacitive membrane ultrasound transducer silicone substrate. The switches 28 are positioned in the probe housing 12 or in an imaging system. The switches 28 selectively connect the voltage source 30 and a ground potential to any of the column electrodes as shown in FIG. 2 or row electrodes in other embodiments. At a given instant in time, the switches 28 are operable to connect one or more electrodes 22, such as one or more of the columns A through F, and disconnect at least another of the electrodes 22, such as one or more of the columns A through F, from the voltage source 30. For example, electrodes 22 associated with columns B, C and D are connected with the voltage source 30 and columns A, E and F electrodes 22 are connected with ground. The connection of the electrodes 22 for columns B, C and D provides an elevation extent of an aperture 18 formed on the array 16 at a given time. The elements 20 along the columns B, C and D are activated by connection to the voltage source and the elements 20 along the columns A, E and F are deactivated by connection to ground. Other connections may be used for activation or deactivation of elements. Non-contiguous apertures may be provided in other embodiments, such as activating elements 20 associated with electrodes 22 along columns B and D and not column C. In yet other embodiments, only a portion of a column or row is activated, such as activating elements in column B extending from row 2 to 4. Electrodes associated with 1, 2 or any number of elements 20 may be provided. The switches 28 are operable to selectively activate and deactivate individual elements 20 or groups of elements 20. Conductors extending from the array to an imaging system are provided for controlling the switches and providing one or more of DC voltages and ground potential. Alternatively, a conductor for each of the electrodes 22 along the rows or columns is provided from the array 16 to the imaging system.

For scanning different imaging planes for real time, near real time or non-real time three-dimensional imaging, the position of the aperture 18 is changed. Different elements 20 are activated, and different elements 20 are deactivated. For example, the example aperture 18 of columns B, C and D is repositioned to instead activate columns D, E and F. The DC voltage source or other activation potential is connected to columns D, E and F, and ground or other inactivation potential is applied to columns A, B and C. Other step sizes of the aperture may be used, such as shifting the aperture by one column, two columns, three columns or any other number of overlapping or non-overlapping columns. While described above as switching the aperture using columns, the control of the aperture may be provided by rows or other arrangement of electrodes 22. Deactivation of certain elements 20 determines an elevation extent of a given aperture 18. The aperture size corresponds to the number of activated electrodes 20. Using the switches 28, the aperture 18 is moved electronically along an elevation dimension or across the array 16. Bias signals are used to selectively address specific portions of the array 16. By selectively moving the aperture 18, a two-dimensional imaging plane is moved through an interrogating volume for forming a three-dimensional image.

In one embodiment, the size of the active aperture 18 is maintained throughout different positions of the overlapping or non-overlapping apertures 18 across the extent of the entire or a portion of the array 16. In alternative embodiments, the aperture size, such as the elevation width corresponding to a number of activated rows, varies as a function of the position of the aperture 18 on the array 16. For example, the elevation extent of the aperture 18 is narrower near the edges of the array 16 than at the center.

One of a transmitter 24, a receiver 26 or combinations thereof connects with other electrodes 22. The transmitters 24 are waveform generators, transistors, switch networks, digital-to-analog converters, waveform generators or other now known or later developed transmitters used in ultrasound transmit beamformation. The receivers 26 are ultrasound receive beamformer channels. In a combination embodiment, a transmit/receive switch connects the transmitters 24 and receivers 26 to each of the channels connected with a separate electrode 22. The transmitters 24 generate relatively phased and apodized waveforms for two-dimensional imaging, and the receivers 26 receive signals from different elements 20 or groups of elements 20 for applying apodization and delays in two-dimensional receive beamformation. In one embodiment, the receive signals or the transmit signals are responsive to each element 20 of the array, but have a much greater or entire contribution from activate elements 20. The inactive elements 20 have minimal or no contribution. For example, the electrodes 22 along the rows extend across the entire array 16, including the active elements 20 or aperture 18. Using the elements 20 as a linear or phased array along the active aperture, two-dimensional ultrasound beamformation is provided for generating an image or signals representing a two-dimensional region. The elements 20 spaced along different rows act as a phased or linear array. As the aperture or elevation position is changed, the transmit and receive acquisition is performed without additional switching of channels to different elements 20. Alternatively, switching is provided for selecting specific elements or groups of elements 20. Using the elements 20 as a linear or phased array, the selected aperture 18 is used to focus a beam in two dimensions. By repositioning the aperture 18 as discussed above, a different two-dimensional plane is scanned using the transmitters 24 and the receivers 26. As a result, a three-dimensional volume is scanned sequentially using the matrix configuration of electrodes 22.

FIG. 4 shows a method for imaging with a multi-dimensional array of an intra-patient probe. Different, additional or fewer acts may be provided in the same or different order than shown. A matrix array of elements is used for activating an aperture and performing phased array or linear imaging using the activated aperture.

In act 40, a group of elements of the multi-dimensional of an intra-patient probe are activated. For example, a different DC voltage is applied to one group of elements than another group of elements. For an electrostrictor array, a ground potential is connected to inactive elements, such as a group of elements corresponding to one or more rows. A bias voltage, such as 20-80 volts, is applied to activate other rows or groups of elements. By activating some elements and not others at a given time, an aperture is generated. Page: 15 Alternately, the beam may be moved in elevation by moving an apodization function wherein there are no or fewer inactive elements, but the apodization function includes elements with alternate polarity and/or elements with reduced activity or weight.

In act 42, ultrasound data is acquired with the activated groups of elements while the elements are activated. Transmission and reception beamformation are performed using activated and deactivated groups of elements as an array. A same electrode may connect both active and inactive elements. The active elements transduce between electrical and acoustical energies and the inactive elements provide minimal or no transduction. For example, an orthogonal matrix configuration is provided where a group of columns and rows are activated. The orthogonally spaced rows or columns, respectively, are then used within the activated aperture as elements. By transmitting and receiving from the activated group of elements as an array, a two-dimensional plane is scanned.

Selective activation associated with an annular array or other non-linear grouping of elements may be provided for scanning along a given scan line or within a two-dimensional plane. For example, annular or sector arrays allow activation of a pair of mirror image sectors or pie shaped groupings of elements. A plane normal to the activated aperture is then scanned using the array extending along the diameter of the annular array. By rotating the selected aperture about a center of the array, different scan planes within a three-dimensional volume that intersect at the center of the array are provided.

In act 44, different groups of elements are activated. For example, at least one element is active in a sequential aperture that was inactive in a preceding aperture. As yet another example, the inactive aperture is shifted by deselecting or inactivating a previously activated row or column of elements. Additionally or alternatively, an additional row or column of elements is activated or added to the aperture. By applying different DC or bias voltages as discussed above to different groups of elements, different elements are activated and others inactivated. By selective activation in a sequential order, the aperture is repositioned and may be moved across the entire or a portion of the face of the array.

In act 46, ultrasound data is acquired with each different aperture. Transmission and reception beamforming using activated elements as an array allows for two-dimensional imaging as discussed above.

The process repeats for each desired scan or desired aperture to scan a volume as shown by the loop back from the acquisition of act 46 to the activation of a different aperture in act 44. By sequentially moving the aperture to different positions on the array and acquiring ultrasound data, ultrasound data associated with different planes within a volume is acquired. For example, less than all. the elements are activated as a function of columns or rows. Elements spaced along rows or columns, respectively, are used as an array. In one embodiment, the rows are substantially orthogonal to the columns, allowing for scanning along two-dimensional planes that are parallel with one another and extend along an elevation dimension.

In one embodiment, the activated apertures are of a same size and shape along both the elevation and azimuth dimension. In other embodiments, the aperture varies in size as a function of the aperture position or time. For example, a number of rows are activated for one aperture position, but a different number of rows are activated for a different aperture position. By sequentially activating different combinations of rows, a lesser number of rows may be simultaneously activated for each aperture at the edges of the array and a greater number of rows are simultaneously activated in each aperture at different positions between the edges. Any of various combinations of step sizes or amounts of repositioning of the aperture, number of rows or elements included within an aperture, spacing of the elements or other array characteristics may be used.

For use in catheters or other small elevation dimension probes, adequate sampling is provided by incrementing the aperture steps by one quarter of the aperture width or elevation extent, but greater or lesser aperture step sizes may be used as a function of the desired depth of imaging. For example, with a 45 degree viewing angle in elevation, rows or columns of elements are provided every 0.3 millimeters. An active aperture of 1.8 millimeters or 6 rows or columns is used for a same or each aperture position. In one embodiment, three segments (e.g., three rows or columns) are provided for an aperture for an edge of the array. The next aperture is generated by adding a further segment, such as a row or column. A further aperture is then generated by adding yet another segment. For the next aperture position, yet another segment is added. An aperture of six segments wide is then walked across the array, such as by adding 1 to 3 segments and subtracting a respective 1 to 3 segments from the aperture. Once the opposite elevation edge is approached, reverse reduction in the number of segments is performed down to three segments.

In one embodiment, the activation bias is the same for all segments of each aperture. In other embodiments, an apodization weight is provided. For a three segment aperture, the relative weight is 1.0 for the center segment and 1.67 for the outer segments. For four segments, the relative weight is 1.0 on the two center segments and 1.59 on the two outer segments. For five segments, the relative weight is 1.0 in the three center segments and 1.67 on the two outer segments. For the six segment aperture, the relative weight is 1.0 in the two center segments and 1.64 on the four outer segments. The weight is of the RF response signal strength of each segment. The RF response is set by the bias voltage. To minimize the number of needed bias voltages, a same two bias voltages for activating the elements may be provided, such as a bias voltage to provide a relative 1.0 and 1.65 weight. Other values, numbers of segments, numbers of bias voltages, patterns of application of apodization to the active segments or combinations thereof may be used.

In act 48, an image is generated as a function of the acquired ultrasound data. For example, a three-dimensional representation is rendered from ultrasound data acquired at different aperture positions. For data associated with two different aperture positions, a three-dimensional representation by scanning along two different planes is provided. In other embodiments, ultrasound data representing three or more planes is acquired. A three-dimensional representation is generated as a function of the acquired data and the associated relative spatial positions of the data.

In an additional embodiment, the data associated with different two-dimensional planes is used to perform a synthetic elevation aperture beamforming process, such as focusing in two dimensions along both the azimuthal and elevation position. In yet another additional or alternative embodiment, a data from multiple scans is combined as a spatial compounding with or without synthetic aperture filtering or beamforming to form a two-dimensional representation. In yet another alternative, a plurality of different two-dimensional representations are acquired and displayed sequentially.

In another alternative embodiment, a capacitive membrane ultrasound transducer disclosed in U.S. Pat. No. 6,676,602, the disclosure of which is incorporated herein by reference, is provided with integrated micro-relays intermingled with the capacitive membrane array. The micro-relays are used to form interconnections between the array elements for implementing the activation and deactivation and associated phased array or linear array beamforming discussed above. The micro-relays may be used to select apertures with a minimum number of control lines and associated signals without supplying a variable or switchable interconnection of DC voltages or ground potential. For example, each element is made out of three membranes or micro-electro-mechanical devices dedicated to switching and any number of membranes dedicated to acoustic transduction, the micro-relays are used to connect together a given element to any of its neighboring elements in a hexagonal pattern. Other relative numbers of capacitive membranes or micro-relays may be used. To provide for a lower actuation voltage of the micro-relays, a micro-relay gap height may be smaller than for the transduction membranes. The diameter of the micro-relays may also be reduced to increase the acoustic aperture relative to the total aperture for a given element. Micro-relays are then used to translate the selected aperture as a function of time for scanning along different two-dimensional planes.

Any combination of imaging may be used. For example, three-dimensional representations are formed by imaging using the array with different aperture positions. The volumetric imaging may allow a user to quickly identify a desired position. Two-dimensional imaging is then used by activating a desired aperture for more detailed examination.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1-24. (canceled)

25. A transducer for use in an ultrasound system for medical imaging or therapy, the transducer comprising:
a catheter;
a two-dimensional ultrasonic acoustic array mounted on the catheter; and
switches operable to apply a voltage to selectively activate array elements, wherein at least one array element is free of activation while at least one other element is activated.

26. The transducer of claim 25 wherein the switches are operable to activate elements of the array by row, different rows activated at different times such that a three-dimensional volume is successively interrogated.

27. The transducer of claim 26 wherein an aperture size corresponding to a number of activated rows varies as a function of position on the array.

28. The transducer of claim 26 further comprising:
first and second voltage sources operable to apply different DC voltages at a same time to different rows.

29. The transducer of claim 25 further comprising:
at least one additional ultrasonic acoustic array mounted on the catheter.

30. The transducer of claim 25 further comprising:
a position sensor within the catheter.
